(19) United States
(12) Patent Application Publication
(10) Pub. No.: US 2006/0241042 A1
(43) Pub. Date: Oct. 26, 2006

(54) Calcineurin activators

(75) Inventors: Hiroko Kitamoto, Tsukuba-shi (JP); Tokichi Miyakawa, Higashi-Hiroshima-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: National Institute of Agrobiological Sciences, Tsukuba-shi (JP)

(21) Appl. No.: 11/441,098

(22) Filed: May 26, 2006

Related U.S. Application Data
(62) Division of application No. 10/507,683, now abandoned.

(30) Foreign Application Priority Data
Mar. 22, 2002 (JP) 2002-81415

(51) Int. Cl. C07K 14/39; A61K 38/16; A23B 7/10

(52) U.S. Cl. 514/12; 530/350; 426/53

(57) ABSTRACT

A calcineurin activator, comprising the following protein (a) or (b) as an active ingredient and having the action of increasing intracellular calcium ion concentration through the influx of calcium ions into eukaryotic cells: (a) a killer protein (KLKP), being composed of 3 subunits consisting of amino acid sequences represented by SEQ ID NOS: 2, 3, and 4, respectively, and being produced by Kluyveromyces lactis killer yeast; or (b) a protein, being the same as protein (a) except for differing from protein (a) in that at least one of the 3 subunits consists of an amino acid sequence derived from the amino acid sequence represented by SEQ ID NO: 2, 3, or 4 by deletion, substitution, or addition of 1 or several amino acids, and having Kluyveromyces lactis killer protein (KLKP) activity.

### TECHNICAL FIELD

The present invention relates to the use of a killer protein (KLKP) produced by killer yeast, Kluyveromyces lactis. The present invention specifically relates to a calcineurin activator comprising a killer protein (KLKP) produced by Kluyveromyces lactis killer yeast as an active ingredient and having the action of increasing intracellular calcium ion concentration through the influx of calcium ions into eukaryotic cells. Furthermore, the present invention relates to an agent for inhibiting eukaryotic cell proliferation and an agent for inhibiting the eukaryotic cell cycle, comprising the calcineurin activator.

### BACKGROUND ART

Recently, various studies have been conducted on the yeast (S. cerevisiae) killer system. A K1 killer protein activates TOKl excessively, and destroys cell membrane functions (Ahmed, A. et al., Cell 99, 283-291 (1999)). Furthermore, destruction and delocalization of chitin synthase III confer resistance against K. lactis killer protein (KLKP) on yeast (Jablonowski, D., et al., Yeast 18 p. 1285 (2001)). It has been reported that KLKP is encoded by a gene on a pGKL1 plasmid and consists of 3 subunits: the α subunit, the β subunit, and the γ subunit (Stark M. J. et al., EMBO J., August 5(8), p. 1995-(1986)).

Furthermore, we have previously reported that the action of KLKP is activated by Ca²⁺. However, the mechanism of the killer activity has not yet been elucidated. Moreover, such yeast killer protein has been thought to have killer activity only against yeast, and the industrial applications thereof have been limited to the field of fermentation and the like.

Calcineurin (CaN) is the only dephosphorylation enzyme of eukaryotes which is controlled by a Ca²⁺/calmodulin complex and plays a central role in the signaling system mediated by Ca²⁺,such as concerning cell proliferation, cell differentiation, and regulation mechanisms for transcriptional control. The CaN action mechanism that has been elucidated in yeast represents important information for analyzing CaN functions in higher eukaryotes. A CaN inhibitor has been put to practical use as an immuniosuppressant. CaN possesses various functions and is expected to be applied in medicine or agriculture. CaN functions that have already been elucidated or industrially applied are shown in Table 1.

TABLE 1
CaN actions and industrial applications thereof

| Action revealed using yeast | Action on higher eukaryotes |
|---|---|
| Site of action of | Immunosuppression |
| immunosuppresant = | (practical use of immunosuppressant |
| CaN inhibition | → medical transplantation) |
| Ion homeostasis*1 | CaN inhibition → Side effects of |
| | immunosuppressant |
| | Renal disorder |
| | Hyperlipidemia |
| | Elevated blood pressure |
| Cell cycle suppression*2 | CaN activation |
| Inositol phospholipid | T cell activation |
| metabolism Cell wall | Induction of cancer cell |
| synthesis | differentiation |
| | Apoptosis |
| | Reconstruction of myofibrils |
| | Plants |
| | Temperature.salt stress resistance |

*Reported by collaborator (1. EMBO Journal, 12, p. 4063 (1993); 2 Nature, 392, p.303 (1998))

It has been reported that CaN activated with Ca²⁺ using budding yeast activates a negative regulation factor of the cell cycle engine, so as to cause a delay in the cell cycle (NATURE, 392, p. 303 (1998)).

As described above, a substance having the action of activating or inhibiting calcineurin has been expected to be industrially applied widely as an agent for inhibiting eukaryotic cell proliferation, in medicines, and the like. However, conventionally the number of easily available calcineurin activators has been very small.

### SUMMARY OF THE INVENTION

An object of the present invention is to provide a calcineurin activator comprising a killer protein (KLKP) produced by Kluyveromyces lactis killer yeast as an active ingredient and having the action of increasing intracellular calcium ion concentration through the influx of calcium ions into eukaryotic cells. Furthermore, another object of the present invention is to provide an agent for inhibiting eukaryotic cell proliferation and an agent for inhibiting eukaryotic cell cycle, comprising the calcineurin activator. A composition containing the calcineurin activator can be broadly applied as an agent for inhibiting eukaryotic cell proliferation, an anticancer agent, and the like. Thus, another object of the present invention is to provide such an agent for inhibiting eukaryotic cell proliferation, an anticancer agent, an immunosuppressant, an immunostimulant, a therapeutic agent against diseases relating to memory, and a therapeutic agent against cardiovascular diseases.

We have discovered that a killer protein (KLKP) produced by Kluyverornyces lactis killer yeast increases intracellular calcium ion concentration through the influx of calcium (Ca²⁺) into budding yeast cells, and then activates calcineurin (CaN) so as to induce a delay at the G2 phase of the cell cycle. We have also discovered that KLKP induces suppressed proliferation of plant culture cells, thereby completing the present invention.

Specifically, the present invention relates to a calcineurin activator comprising Kluyveromyces lactis killer protein (KLKP) as an active ingredient and having the action of increasing intracellular calcium ion concentration through the influx of calcium ions into eukaryotic cells.

Furthermore, the present invention relates to the above calcineurin activator comprising one of the following proteins as an active ingredient and having the action of increasing intracellular calcium ion concentration through the influx of calcium ions into eukaryotic cells:
(a) a Kluyveroniyces lactis killer protein (KLKP), consisting of 3 subunits: a subunit consisting of an amino acid sequence represented by SEQ ID NO: 2, a subunit consisting of an amino acid sequence represented by SEQ ID NO: 3, and a subunit consisting of an amino acid sequence represented by SEQ ID NO: 4; or
(b) a protein, being the same as protein (a) except for differing from protein (a) in that at least one of the 3 subunits consists of an amino acid sequence derived from the amino acid sequence represented by SEQ ID NO: 2, 3, or 4 by deletion, substitution, or addition of 1 or several amino acids, and having Kluyveromyces lactis killer protein (KLKP) activity.

Furthermore, the present invention relates to an agent for inhibiting eukaryotic cell proliferation comprising calcium ions and the above calcineurin activator.

Furthermore, the present invention relates to an agent for inhibiting eukaryotic cell cycle comprising calcium ions and the above calcineurin activator.

Furthermore, the present invention relates to an agent for inhibiting eukaryotic cell cycle comprising a prepared product comprising cheese whey or cheese-whey-derived calcium at a high concentration and the above calcineurin activator.

Furthermore, the present invention relates to a method for preventing aerobic deterioration of silage, comprising a step of simultaneously or separately adding calcium ions and the above calcineurin activator to silage.

Furthermore, the present invention relates to the above method, wherein calcium ions are added by adding a prepared product comprising cheese whey or cheese-whey-derived calcium at a high concentration.

Furthermore, the present invention relates to a pharmaceutical composition comprising the above calcineurin activator, which is selected from the group consisting of an anticancer agent, an immunosuppressant, an immunostimulant, a therapeutic agent against disorders relating to memory, and a therapeutic agent against cardiovascular diseases.

Furthermore, the present invention relates to a method for screening for a compound involved in calcineurin inhibition, comprising causing a calcineurin activator comprising Kluyveromnyces lactis killer protein (KLKP) as an active ingredient and having the action of increasing intracellular calcium ion concentration through the influx of calcium ions into eukaryotic cells and a test substance to act on a KLKP-sensitive yeast strain, and using the ability of the test substance to cancel the sensitivity of the KLKP-sensitive cell as an index.

Furthermore, the present invention relates to a method for screening for a remedy against diseases in which calcineurin is involved, comprising causing a calcineurin activator comprising Kluyveromyces lactis killer protein (KLKP) as an active ingredient and having the action of increasing intracellular calcium ion concentration through the influx of calcium ions into eukaryotic cells and a test substance to act on a KLKP-sensitive yeast strain, and using the ability of the test substance to cancel the sensitivity of the KLKP-sensitive yeast as an index.

Furthermore, the present invention relates to the above method, wherein the remedy against diseases in which calcineurin is involved is selected from the group consisting of an anti-cancer agent, an immunosuppressant, an immunostimulant, a therapeutic agent against disorders relating to memory, and a therapeutic agent against cardiovascular diseases.

The present invention is explained in detail as follows.

1. Obtainment of Killer Protein (KLKP) Produced by Kluyveromyces lactis Killer Yeast

Killer yeast means yeast that produces a killer toxin selectively suppressing the growth of other types of yeast. A killer protein KLKP produced by the killer yeast used in the present invention can be obtained from Kluyveromyces lactis (Kitamoto, H. K. et al., (1995), 7^{th} European Congress on Biotechnology, Abstract Book, 62). The killer protein KLKP produced by Kluyveromyces lactis consists of 3 subunits: α, β, and γ, and DNAs encoding these subunits are present on a linear plasmid, pGKL1. An example of Kluyveromyces lactis is the IFO1267 strain. The Kluyveromyces lactis IFO1267 strain can be obtained from the Institute for Fermentation, Osaka (IFO) (2-17-85, Jusohhommachi, Yodogawa-ku, Osaka-shi). The above killer yeast is cultured, and then KLKP can be isolated and purified from the culture product. Here, the culture product means a culture supernatant and cultured microbial bodies. Yeast can be cultured by known methods. Moreover, KLKP can be isolated and purified from the culture product using known biochemical techniques using, for example, a hydroxyapatite column. Whether or not a purified protein is KLKP can be determined by measuring if the protein has yeast killer activity.

Moreover, a gene encoding KLKP is isolated from Kluyveromyces lactis, and then a recombinant KLKP can be obtained by genetic engineering techniques.

Experiments required for obtaining recombinant KLKP, such as mRNA preparation, cDNA production, the RT-PCR method, the RACE method, determination of the nucleotide sequence of DNA, and examination of expression by Northern blot can be conducted by methods described in general experimental manuals such as the one edited by Sambrook et al (Molecular Cloning, A laboratory manual, 2001, Eds., Sambrook, J. & Russell, D W. Cold Spring Harbor Laboratory Press).

The DNA sequence of the pGKL1 linear plasmid wherein KLKP is present is shown in SEQ ID NO: 1. The DNA has been registered with GenBank under accession number X00762. Of these, a sequence between positions 3229 and 6669 encodes the large subunit of the killer protein. Among the amino acid sequences of the large subunit, an amino acid sequence between positions 30 and 892 corresponds to the α subunit, and an amino acid sequence between positions 895 and 1146 corresponds to the β subunit. A sequence between positions 7939 and 8688 of SEQ ID NO: 1 encodes the small subunit of KLKP, that is, the γ subunit. The amino acid sequences of the α, β, and γ subunits of KLKP are shown in SEQ ID NOS: 2, 3, and 4, respectively.

A protein that is also included in examples of the protein of the present invention is a protein consisting of the α, β, and γ subunits represented by SEQ ID NOS: 2, 3, and 4, respectively, wherein: at least 1 or preferably 1 or several (e.g., 1 to 10 and further preferably 1 to 5) amino acids may be deleted from the amino acid sequence of at least one of the 3 subunits; at least 1 or preferably 1 or several (e.g., 1 to 10 and further preferably 1 to 5) amino acids may be added to the amino acid sequence represented by SEQ ID NO: 2; or at least 1 or preferably 1 or several (e.g., 1 to 10 and further preferably 1 to 5) amino acids may be substituted with other amino acids, and having killer yeast activity or the action of activating calcineurin.

An example of such an amino acid sequence derived from the amino acid sequence represented by SEQ ID NO: 2, 3, or 4 by deletion, substitution, or addition of 1 or several amino acids is an amino acid sequence having at least 80% or more, preferably 90% or more, more preferably 95% or more, and particularly preferably 97% or more homology, with the amino acid sequence of SEQ ID NO: 2, 3, or 4 when calculation is conducted using BLAST or the like (for example, when calculation is conducted with default, that is, initial setting conditions). A protein consisting of 3 subunits having such homologies and having killer yeast activity or the action of activating calcineurin is also included among the examples of the protein of the present invention.

Here, killer yeast activity means activity suppressing the proliferation of wild yeasts and can be assayed using as an index the suppression of the proliferation of yeast when yeast is cultured with the protein of the present invention added to the yeast. For example, killer yeast activity can be assayed by a method that involves culturing yeast in a medium on which a paper disk containing the protein of the present invention is placed, and measuring the area of the inhibition zone.

Furthermore, the action of activating calcineurin means the action of increasing the intracellular calcium ion level so as to activate calcineurin, the calcium-signaling pathway. This activation action can be measured by the method described below.

The gene is incorporated into an appropriate vector, and then an appropriate host is transformed with the vector, so that a recombinant KLKP can be expressed and obtained.

The recombinant vector of the present invention can be obtained by ligating (inserting) the gene of the present invention into an appropriate vector. Examples of a vector to be used for the insertion of the gene of the present invention are not specifically limited, as long as such vectors can be replicated in a host, and include a plasmid DNA and a phage DNA.

An example of a method that is employed for inserting the gene of the present invention into a vector involves, first, cleaving a purified DNA with an appropriate restriction enzyme, and then inserting the cleaved product at a restriction enzyme site or a multicloning site of an appropriate vector DNA, so as to ligate the product to the vector.

It is necessary for the gene of the present invention to be incorporated into a vector so that the gene functions can be exerted. Hence, in addition to a promoter and the gene of the present invention, if desired, a sequence containing a cis element such as an enhancer, a splicing signal consisting of a splice donor site located on the 5′ terminal side of an intron and a splice receptor site located on the 3′ terminal side of the intron, a polyA addition signal, a selection marker, a ribosome binding sequence (SD sequence), and the like can be ligated to the vector of the present invention.

A transformant of the present invention can be obtained by introducing the recombinant vector of the present invention into a host so that a target gene can be expressed. Here, an example of a host is not specifically limited, as long as it can express the DNA of the present invention. Examples of such a host include: bacteria belonging to the genus Escherichia such as Escherichia coli, the genus Bacillus such as Bacillus subtilis, and the genus Pseudomonas such as Pseudomnonas putida; yeast such as Kluyveromyces lactis, Saccharomyces cerevisiae, and Schizosaccharoniyces pombe; animal cells such as COS cells and CHO cells; and insect cells such as S121.

Examples of a method for introducing a recombinant vector into bacteria are not specifically limited, as long as such methods are methods for introducing DNA into bacteria. Examples thereof include a method using calcium ions [Cohen, S. N. et al., Proc. Natl. Acad. Sci., U.S.A., 69, 2110 (1972)] and electroporation.

The protein of the present invention can be obtained by culturing the above transformant and then collecting the protein from the culture product. The “culture product of the transformant” means any of a culture supernatant, cultured cells, cultured microbial bodies, and disrupted products of cells or microbial bodies.

After culture, when the protein of the present invention is produced within microbial bodies or cells, KLKP is extracted by disrupting the microbial bodies or cells. In addition, when the protein of the present invention is produced outside of microbial bodies or cells, the broth is directly used or the broth is subjected to centrifugation or the like to remove the cells. Subsequently, through the use of one of or an appropriate combination of general biochemical methods employed for isolation and purification of proteins, such as ammonium sulfate precipitation, gel chromatography, ion exchange chromatography, and affinity chromatography, the protein of the present invention can be isolated and purified from the above culture product. In a manner similar to purification from Kluyveromyces lactis killer yeast, it is preferable to conduct purification by chromatography using hydroxyapatite gel.

2. Use of Killer Protein (KLKP) Produced by Kluyveromyces lactis Killer Yeast as Calcineurin Activator

KLKP purified from the above culture product of Kluyveromyces lactis killer yeast or KLKP obtained by genetic engineering techniques can be used as the calcineurin (CaN) activator of the present invention.

The killer protein that is produced by Kluyveromyces lactis killer yeast increases the calcium concentration within eukaryotic cells through the influx of calcium ions into the cells so as to activate calcineurin, thereby inducing a delay at the G2 phase of the cell cycle.

Here, the influx of calcium ions into cells includes not only the influx of calcium ions existing extracellularly into cells, but also the influx of calcium ions existing in the organelles within cells into cytoplasms. Hence an increase in the intracellular calcium ion concentration means an increase in the intracytoplasmic calcium ion concentration.

The calcineurin activator of the present invention comprising KLKP as an active ingredient can be used as a reagent for studying the signaling system mediated by Ca²⁺, such as cell proliferation, cell differentiation, and regulatory mechanisms for transcriptional control in eukaryotes. Moreover, the activation of calcineurin induces a delay at the G2 phase of the cell cycle. Thus, the calcineurin activator of the present invention can be used as an agent for inhibiting the cell cycle of eukaryotic cells such as yeast, plant, and animal cells. Furthermore, the calcineurin activator of the present invention inhibits the cell cycle, so that it can be used as an agent for inhibiting cell proliferation. Furthermore, the activation of calcineurin results in T-cell proliferation, induction of cancer cell differentiation, apoptosis, and reconstruction of myofibrils. Thus, the calcineurin activator can also be used as an agent for activating the immune system, an anti-cancer agent, or the like.

The action of KLKP to activate calcineurin can be confirmed as described below.

Based on the fact that the calcium-signaling pathway activates Swe1, the action of KLKP can be confirmed using changes in killer sensitivity when the calcium-signaling pathway is blocked.

Moreover, the activation of calcineurin, the calcium-signaling pathway, can also be confirmed using the fact that when KLKP is caused to coexist with calcium in Δzds1 (Zds1-disrupted strain), the cell cycle is delayed at the G2 phase, as an index. Delay at the G2 phase of the cell cycle can be confirmed by FACS analysis.

3. Use of the Killer Protein (KLKP) Produced by Kluyveromyces lactis Killer Yeast as Agent for Inhibiting Cell Proliferation

The Killer protein (KLKP) produced by Kluyveromyces lactis killer yeast of the present invention can be used as an agent for inhibiting cell proliferation.

KLKP can be used as an agent for inhibiting cell proliferation by causing KLKP to come into contact with cells, or to be expressed within cells.

For example, KLKP can be used as an antifungal agent or the like for suppressing mycetes proliferation. In addition, KLKP inhibits cell proliferation, so that it can also be used as an agent for inhibiting the proliferation of an organism body itself, such as plant bodies. The type of KLKP to be caused to come into contact with cells or organism bodies can be appropriately determined depending on the species and quantities of organisms whose proliferation is to be inhibited by KLKP. For example, in the case of cultured tobacco cells, KLKP is added at a final concentration between 1/100 and 1/1000 to a broth supplemented with cells where the ratio of cells to solution by live weight is 1/40.

4. Prevention of Deterioration of Silage (Feed of Cattle) Using Killer Protein (KLKP) Produced by Kluyveromyces lactis Killer Yeast

Silage is feed of ruminant domestic animals having an improved preservative quality, which is produced by lactate fermentation of grass. When silage is exposed to an aerobic condition, aerobic deterioration proceeds due to the lactate metabolism of yeast. To prevent such aerobic deterioration, it is necessary to suppress the proliferation of lactic-acid-assimilating wild yeasts that cause deterioration.

The killer protein (KLKP) produced by Kluyveromyces lactis killer yeast suppresses the proliferation of fungal cells such as yeast in the presence of calcium ions. Thus, KLKP can be used for preventing silage deterioration. Per kg of silage, 0.1 g to 0.5 g of calcium ions and 0.1 g to 0.5 g of KLKP may be added. In addition, as a supply source of calcium ions, for example, cheese whey can be used. Calcium ions and KLKP may be added simultaneously or separately to silage.

A composition comprising calcium ions or a supply source of calcium ions and KLKP for preventing silage deterioration is also encompassed within the scope of the present invention.

5. Use of Killer Protein (KLKP) Produced by Kluyveromyces lactis Killer Yeast as Medicine

A calcineurin activator comprising KLKP as an active ingredient can also be used as a medicine. Since calcineurin has various functions, activation of calcineurin can be used for preventing and treating various diseases.

The calcineurin activator of the present invention comprising KLKP as an active ingredient can be utilized as, for example, an anti-cancer agent. Furthermore, the calcineurin activator of the present invention comprising KLKP as an active ingredient can also be utilized as an immunosuppresant, an immunostimulant, a therapeutic agent against disorders relating to memory, and a therapeutic agent against cardiovascular diseases. A pharmaceutical composition containing the calcineurin activator comprising KLKP as an active ingredient is also encompassed within the scope of the present invention. Here, examples of disorders relating to memory include hypomnnesia, amnesia, anterograde amnesia, and emotional amnesia. Examples of cardiovascular diseases include ischemic neuronopathy.

A method for treating disorders relating to memory, a method for treating cardiovascular diseases, and a method for suppressing or activating immunity by administering the calcineurin activator of the present invention comprising KLKP as an active ingredient to patients are also encompassed within the scope of the present invention. Moreover, the use of the calcineurin activator of the present invention comprising KLKP as an active ingredient in production of an anti-cancer agent, an immunosuppresant, an immunostimulant, a therapeutic agent against disorders relating to memory, or a therapeutic agent against cardiovascular diseases is also encompassed within the scope of the present invention.

A medicine of the present invention can be administered in various forms. Examples of such forms for administration include tablets, capsules, granules, powder, syrup, and the like used for oral administration, and injections, drops, suppositories, and the like for parenteral administration. Such a composition is produced by known methods, and contains a carrier, a diluent, and an excipient that are generally used in the field of pharmaceutical preparations. For example, as a carrier and an excipient for a tablet, lactose, magnesium stearate, or the like is used. An injection is prepared by dissolving, suspending, or emulsifying KLKP or a salt thereof in a sterile aqueous or oil solution that is generally used for an injection. As an aqueous solution for injection, physiological saline, an isotonic solution containing glucose and other adjuvants, or the like is employed. The aqueous solution for injection can be used together with an appropriate solubilizer, for example, alcohol, polyalcohol such as propylene glycol, or a nonionic surface active agent. As an oil solution, sesame oil, soybean oil, or the like is used. As a solubilizer, benzyl benzoate, benzyl alcohol, or the like may be used together.

The dose differs depending on symptom, age, body weight, and the like, and ranges from approximately 0.001 mg to 100 mg per day in general oral administration. Administration is conducted once or several separate times per day. In the case of parenteral administration, a dose ranging from 0.001 mg to 100 mg per administration is conducted by subcutaneous injection, intramuscular injection, or intravenous injection.

Furthermore, the calcineurin activator of the present invention comprising Kluyveromyces lactis killer protein (KLKP) as an active ingredient and having the action of increasing intracellular calcium ion concentration through the influx of calcium ions into eukaryotic cells can be used for screening for a remedy against diseases in which calcineurin is involved. For example, screening can be conducted as described below.

When a KLKP-sensitive strain such as Δzds1(Zds1-disrupted strain) is placed in a microplate together with a medium and calcium, and then KLKP is added to the microplate, or recombinant KLKP is expressed, proliferation is generally inhibited. If proliferation is not inhibited when various drugs are added, the drugs can be determined to be calcineurin inhibitors.

This screening can be conducted using not only yeast, but also animal cells.

### BRIEF DESCRIPTION OF THE DRAWINGS

FIG. 1 shows the results of searching killer proteins that are activated by calcium.

FIG. 2 shows the killer effect of KLKP against the W303 strain. (a) shows the results when Ca²⁺ coexisted with KLKP and (b) shows the results when other ions coexisted with KLKP.

FIG. 3 shows the killer effect against Ca²⁺ signaling system variants. FIG. 3A shows the results for (a) the parent W303 strain and (b) the zds1 gene-disrupted strain (Δzds1) thereof. FIG. 3B shows the results for double gene (including Δzds1)-disrupted strains. (c) shows the results for Δzds1Δswe1, (d) shows the results for Δzds1Δcnb1, and (e) shows the results for Δzds1Δmpk1.

FIG. 4 shows the G2/M-phase cell cycle model participating in a budding yeast Ca²⁺ signaling system.

FIG. 5 shows the results of FACS analyses conducted for the Δzds1 strain (Zds1-destrupted strain) on which KLKP was caused to act.

FIG. 6 shows the results of FACS analyses conducted for double (Δzds1 and Ca²⁺ signaling system) variants on which KLKP was caused to act. (a) shows the results for Δzds1Δcnb1, (b) shows the results for Δzds1Δswe1. and (c) shows the results for Δzds1Δmpk1.

FIG. 7 shows the effect of KLKP on viable count. (a) shows the results for the parent W303 strain, and (b) shows the results for Δzds1.

FIG. 8 shows photographs showing the appearance of (a) chitin (9 hours later) and (b) actin (6 hours later) of cells on which KLKP was caused to act.

FIG. 9 shows emission levels in wild-type cells and cells caused to express aequorin (FIG. 9(a)) and an increase in intracellular calcium level by KLKP (reporter assay method) (FIG. 9(b)).

FIG. 10 shows the result of analyzing the action of KLKP using ⁴⁵Ca.

FIG. 11 shows photographs showing the growth of cultured tobacco cells on which KLKP was caused to act.

### BEST MODE OF CARRYING OUT THE INVENTION

The present invention will be further described specifically by referring to examples, but the technical scope of the present invention is not limited by these examples.

### EXAMPLE 1

### Purification of Killer Protein (KLKP) Produced By Kluyveromyces lactis Killer Yeast.

100 ml of YPD medium (yeast extract 1%, peptone 2%, and glucose 2%) in which the Kluyveromyces lactis yeast IFO1267 strain had been inoculated was put in a 500 ml flask, and then the yeast was subjected to rotation and shake culture overnight at 28° C. and 220 rpm. 1.3 L of the broth was centrifuged. The resulting supernatant was filtered with a 0.2 μm filter, and then concentrated to 10 ml using a ultrafiltration system (Asahi Kasei Corporation. ACP-1010 and SLP-0053). After substitution with a 10 mM potassium phosphate buffer (pH 6.8), the resliltant was adsorbed to a hydroxyapatite column (Nacalai Tesque, Inc., 187-37, 100-200 mesh) that had been equilibrated with the same buffer. After washing with a 10 mM potassium phosphate buffer (pH 6.8), the killer protein was eluted using a 400 mM potassium phosphate buffer (pH 6.8). Fractions having killer activity were collected, dialyzed using a 50 mM citrate-phosphate buffer (pH 6.0), and then filtered and sterilized using a 0.2 μm filter. Glycerol was added at a final concentration of 10% to 10 ml, and then the resultant was stored at −80° C.

### EXAMPLE 2

### Increase in Intracellular Ca²⁺ Level and Activation of Calcineurin by Killer Protein (KLKP) Produced by Kluyveromyces lactis Killer Yeast

9 types of killer yeast culture filtrates were caused to act on budding yeast in the presence of Ca²⁺, and then growth inhibition was examined. The 9 types of killer yeasts were the Saccharonmyces cerevisiae NCYC235 strain (hereinafter, the name of a killer type is K1), the Saccharomyces cerevisiae NCYC738 strain (K2), the Saccharonmyces cerevisiae NCYC761 strain (K3), the Candida glabrata NCYC388 strain (K4), the Pichia anomala NCYC434 strain (K5), the Kluyveromyces arxianus NCYC587 strain (K6), the Candida valida NCYC327 strain (K7), the Kluyveromyces lactis NCYC575 strain (K10), and the Williopsis saturnus var. saturnus IFO0117 strain. Furthermore, proliferation of cell cycle-related gene variants in the presence of various metal salts was examined on a 96-well microplate using KLKP that had been purified using the hydroxyapatite column from the killer yeast culture filtrate. Similarly, after shake culture within a flask in the presence of 50 mM Ca²⁺ and 1/100 KLKP, viable count was examined with time. Furthermore, the cell cycles of immobilized cells were examined using a flow cytometry system (FACS), thereby analyzing the morphology, nuclei, actin, and chitin by fluorescence microscope observation. As a result, the following matters were revealed.

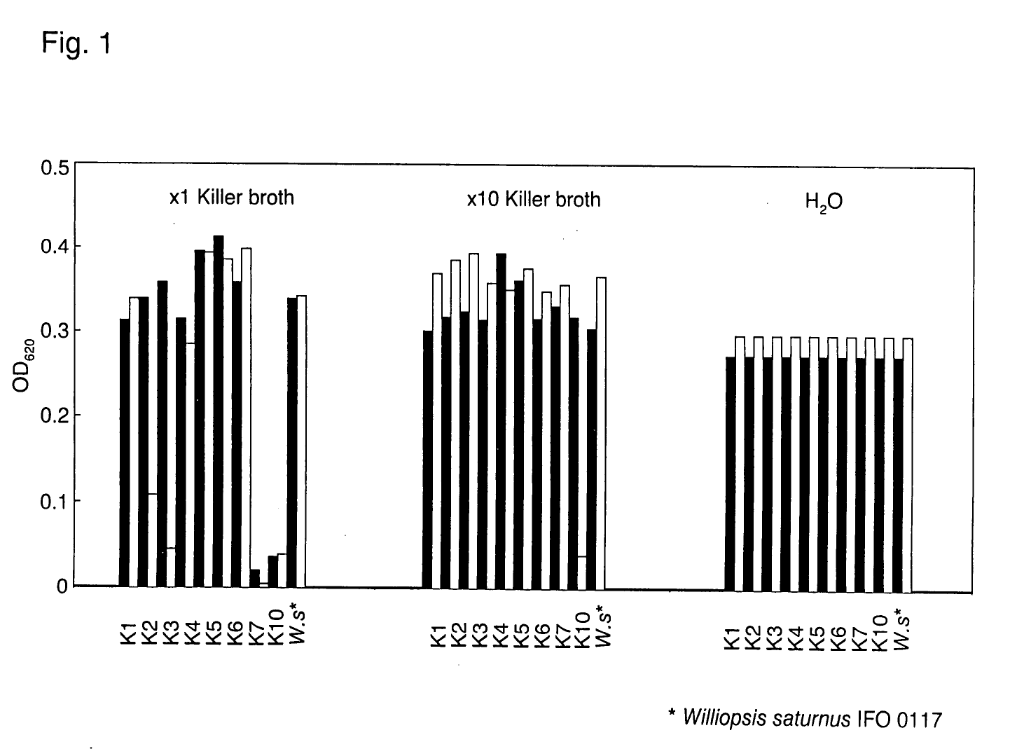

Of the killer yeast strains tested, 4 strains (the Saccharomyces cerevisiae NCYC738 strain (K2), the Saccharomyces cerevisiae NCYC761 strain (K3), the Candida valida NCYC327 strain (K7) and the Kluyveromyces lactis NCYC575 strain (K10)) inhibited the proliferation of budding yeast while coexisting with Ca²⁺. However, the Kluyveromyces lactis broth used in this study showed strong activity of inhibiting proliferation, even if it had been diluted 10-fold, a dilution at which no other killer yeasts showed such activity (FIG. 1). In FIG. 1, white bars indicate cases where calcium was present, and black bars indicate cases where no calcium was present. The horizontal axis indicates the degree of proliferation.

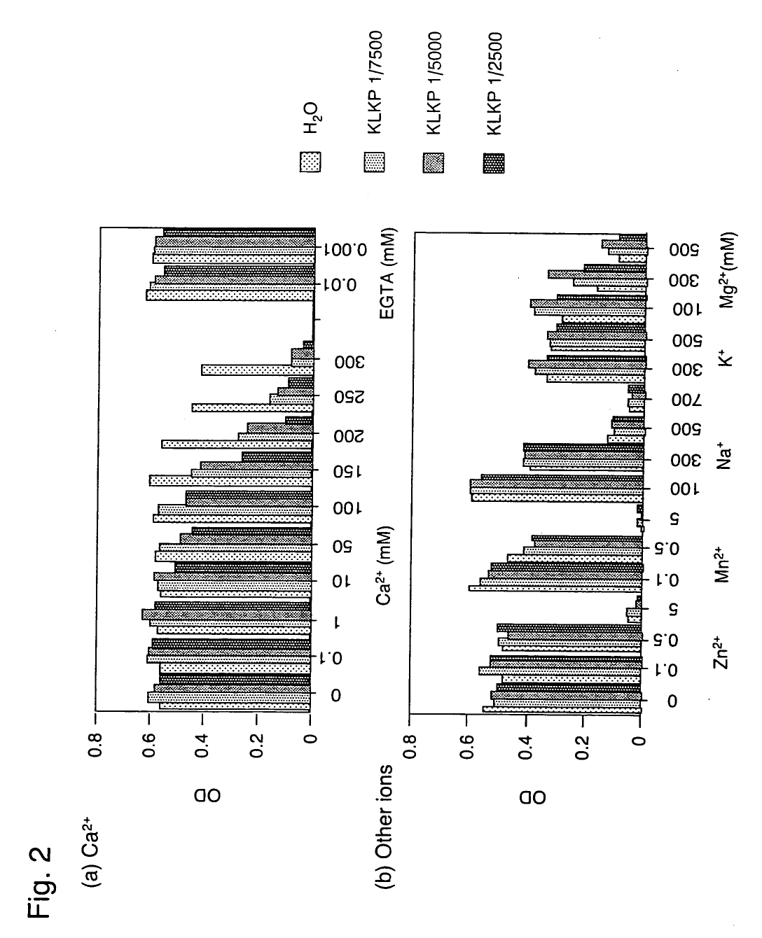

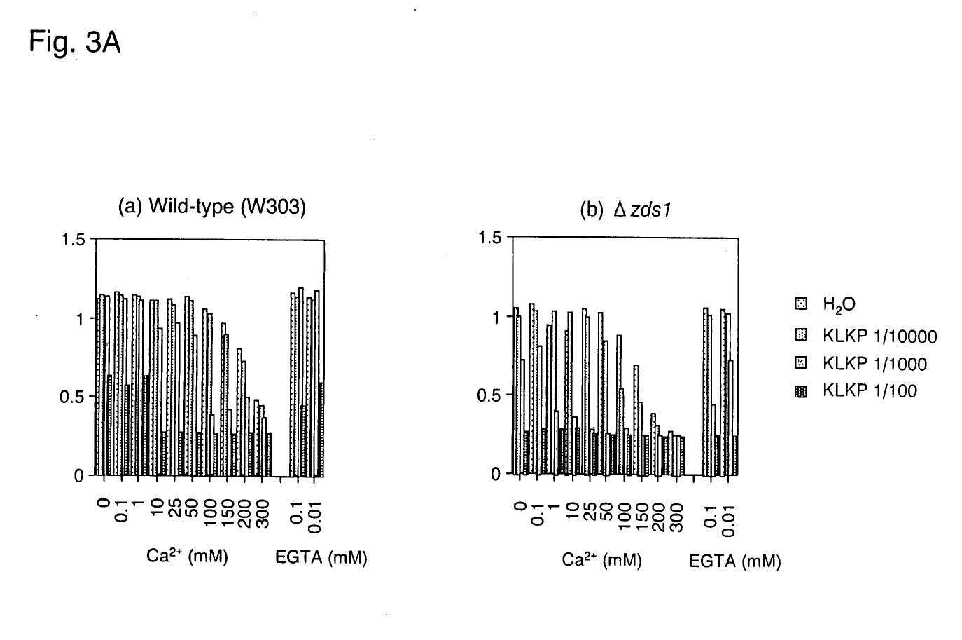

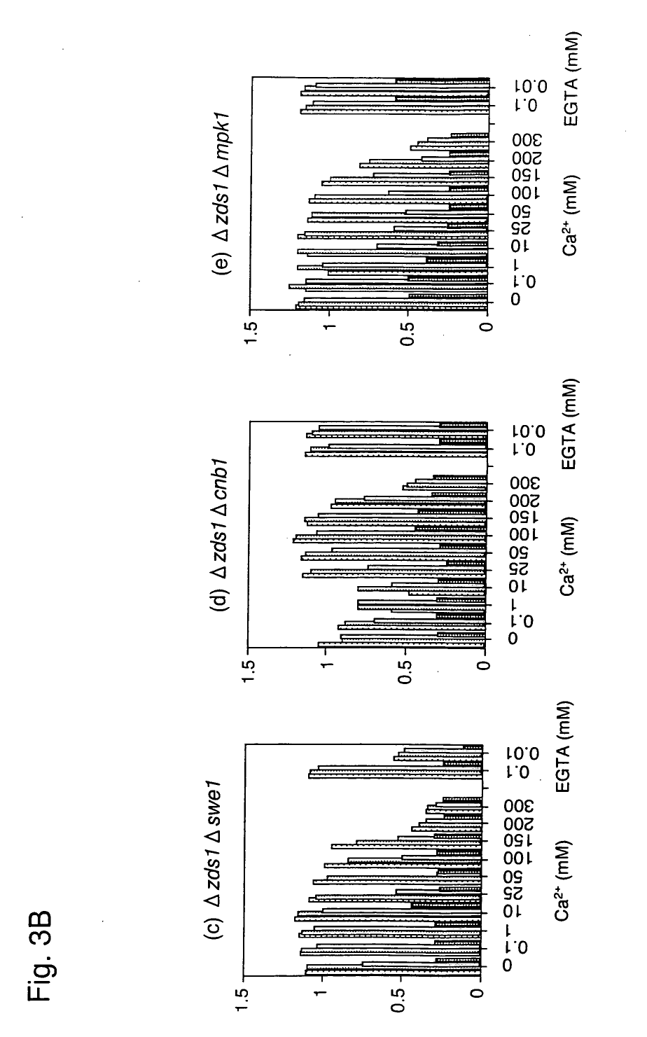

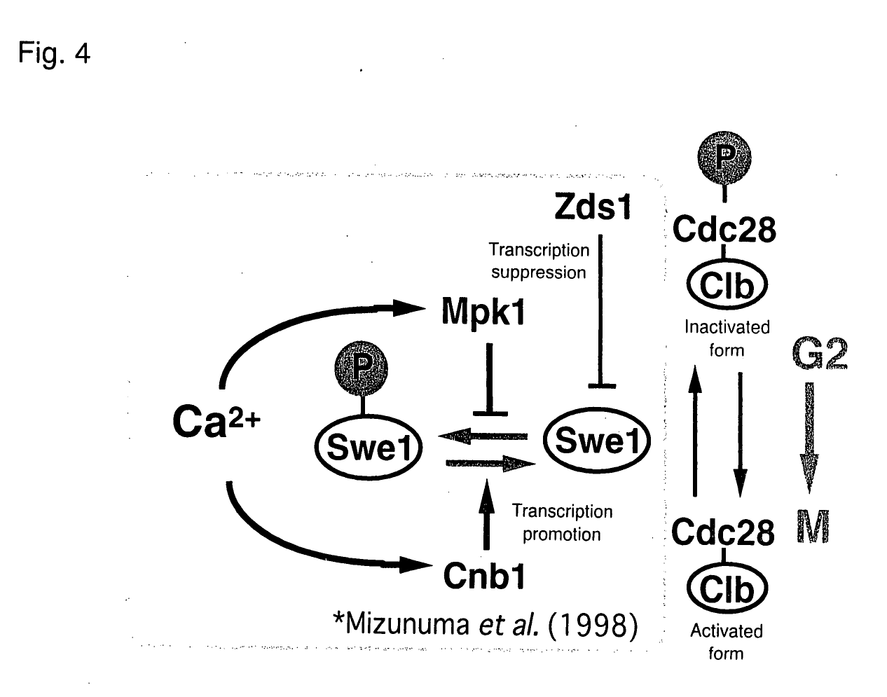

Furthermore, the proliferation of the budding yeast W303 strain was inhibited by KLKP at high Ca²⁺ concentrations, but was not affected by Zn²⁺, Mn²⁺, Na⁺, K⁺, or Mg²⁺ (FIGS. 2(a) and (b)). The Δzds1 strain (Zds1-desrupted strain) showing sensitivity to high concentrations of Ca²⁺, which had been isolated from the W303 strain, showed sensitivity to KLKP at concentrations lower than those to which the W303 strain showed sensitivity. Furthermore, the growth inhibition of the Δzds1 strain by KLKP was suppressed by the addition of EGTA, a Ca²⁺ chelating agent (FIG. 3(b)). Zds1 suppresses the transcription of Swe1, which is the negative regulation factor of the cell cycle engine. In Δzds1, Swe1 is transcribed and the Cdc28-Clb cell cycle engine is phosphorylated, so that the Δzds1 strain cell cycle is delayed at the G2 phase. In addition, Ca²⁺ signaling system calcineurin and Mpk1 MAP kinase transcribe and activate Swe1, respectively in the presence of Ca²⁺, thereby negatively controlling the cell cycle engine (FIG. 4). The mechanism shown in FIG. 4 has been reported by Mizunuma et al (NATURE, 392, p. 303 (1998)). Hence, in Δzds1, the effect of Ca²⁺ on cell cycle can be observed. The double gene (Δzds1 and Ca²⁺ signaling system)-disrupted strains Δzds1Δswe1, Δzds1Δcnb1 (calcineurin subunit), and Δzds1Δmpk1 showed KLKP sensitivity lower than that of Δzds1 at 0.1 mM to 300 mM Ca²⁺⁺ (FIGS. 3(c), (d), and (e)).

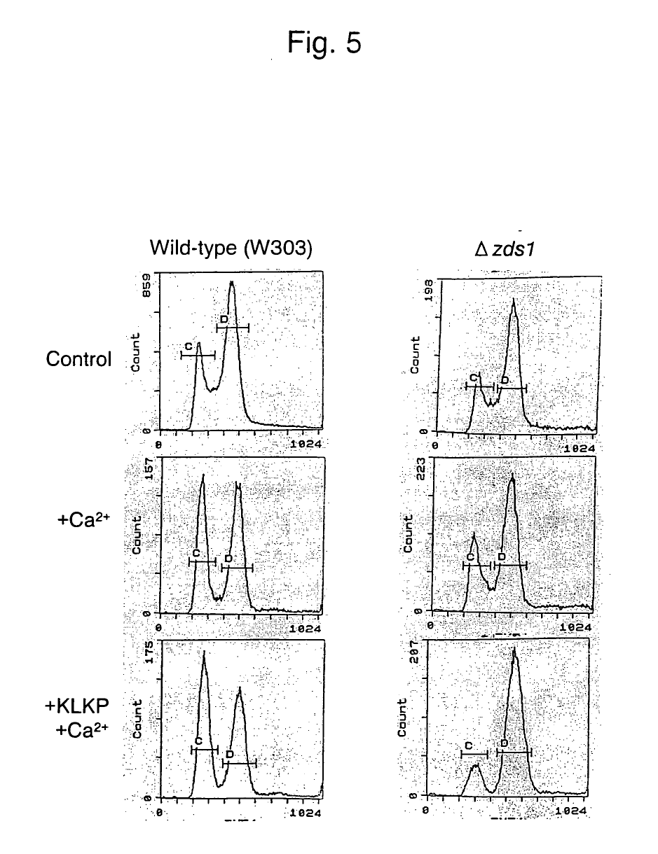

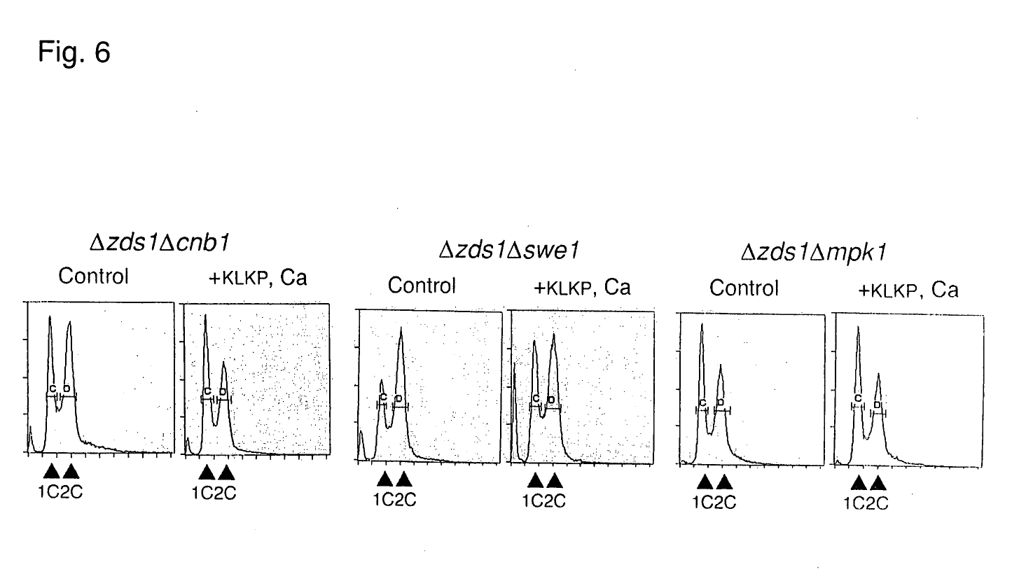

As a result of FACS analyses, G2-phase cells increased in the Δzds1 strain by the addition of KLKP (FIG. 5). In the meantime, in Δzds1Δcnb1 and Δzds1Δswe1, G6-phase cells increased by the addition of KLKP, and in Δzds1Δmpk1, G2-phase cells did not increase as in the case where no KLKP had been added (FIG. 6). Regarding the 2 peaks in the results of FACS analyses in FIGS. 5 and 6, the 1^{st} peak shows an increase in cells at the G1 phase, and the 2^{nd} peak shows an increase in cells at the G2 phase.

These results revealed that the addition of KLKP results in a delay at the G2 phase of the cell cycle of budding yeast, and this action is activated by Ca²⁺. In this mechanism, activation of Swe1 kinase by a Ca²⁺ signaling pathway is involved. Although KLKP is known to arrest the cell cycle of budding yeast at the G1 phase, it has been reported that a high concentration of KLKP often arrests the cell cycle at a different phase. Hence, it was concluded that suppression of the cell cycle by KLKP takes place at two phases (the G1 phase and the G2 phase), and the mechanism activated by Ca²⁺ causes a delay at the G2 phase. When the Ca²⁺ signaling pathway is blocked, a suppressed cell cycle is observed at the other G1 phase as observed in FIG. 6.

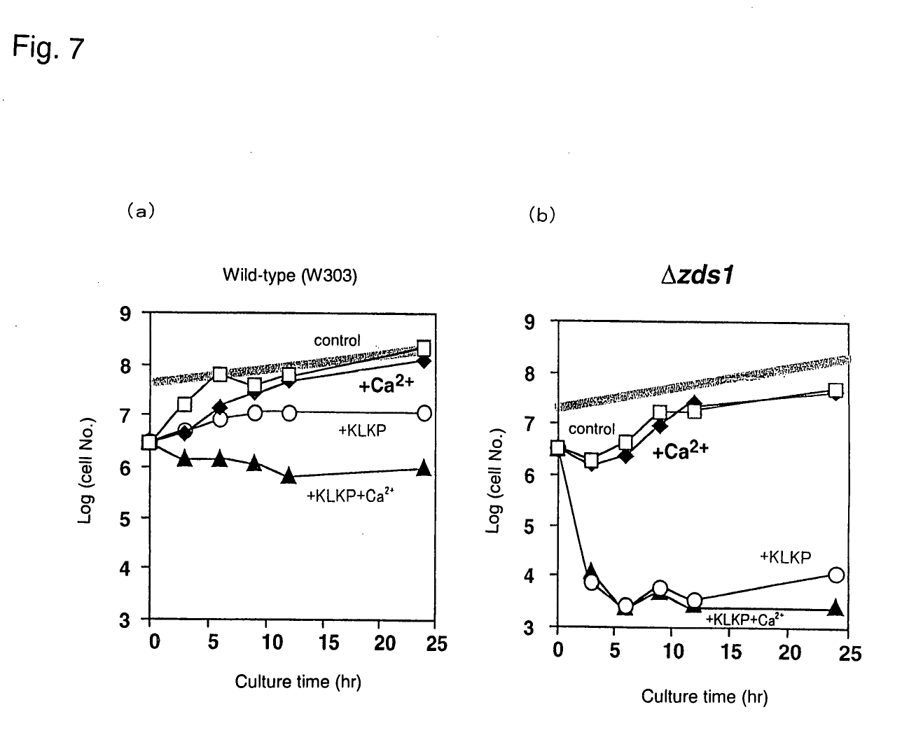

Under the culture conditions, the viable count of the W303 strain was hardly ever affected. However, the Δzds1 strain rapidly died due to the addition of KLKP, showing hypersensitivity to KLKP (FIGS. 7(a) and (b)). The gray lines in FIG. 7 show the total number of microbial bodies. In addition, FIG. 7(a) shows the result for the parent W303 strain, and FIG. 7(b) shows the result for the Δzds strain.

Under the culture conditions, the viable count of the W303 strain was hardly ever affected. However, the Δzds1 strain rapidly died by the addition of KLKP, showing hypersensitivity to KLKP (FIG. 7).

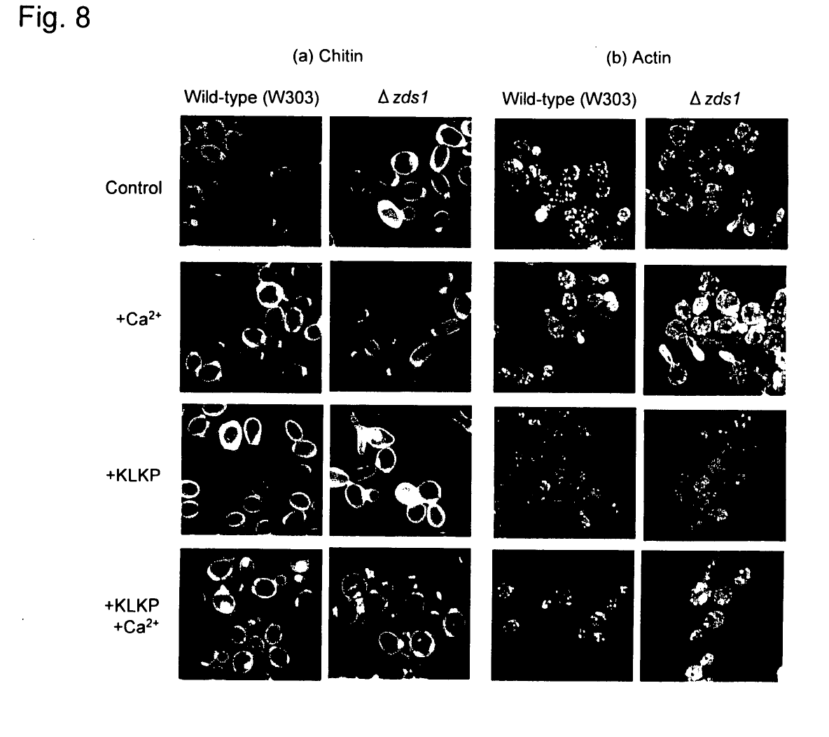

KLKP is known to recognize chitin. Chitin is generally localized at bud scars. However, in both the cases of the W303 strain and the Δzds1 strain, chitin masses were scattered on the cell cortex due to the addition of Ca²⁺ and KLKP (FIG. 8(a)). Moreover, in the case of actin, it is known that cables generally run in the direction of budding, that patches are localized at budding positions, and that Ca²⁺ has an effect on actin localization. In both the cases of the W303 strain and Δzds1 strain, actin localization became deficient due to the addition of KLKP. Deficiency in chitin and actin localization due to KLKP was enhanced by Ca²⁺ coexisting with KLKP, and this was more serious in the Δzds1 strain than in the W303 strain (FIG. 8(b)). As described above, KLKP generally exerts a bacteriostatic (fungistatic) effect, inducing deficiency in actin localization. Furthermore, KLKP also induces deficiency in chitin localization of KLKP-hypersensitive strains due to the activation of KLKP by Ca²⁺. In addition, it was observed that KLKP kills cells.

Furthermore, intracellular calcium concentrations increased by KLKP were measured.

Yeast that had been caused to express a luminescent protein aequorin precursor was cultured in a medium containing no calcium ions. The yeast was caused to absorb a substrate (coelenterazine), Ca²⁺ and KLKP were caused to act thereon, and then emission levels were measured using a photon counter. In addition, a calcium ionophore (A23187) was used as a control of Ca²⁺ influx.

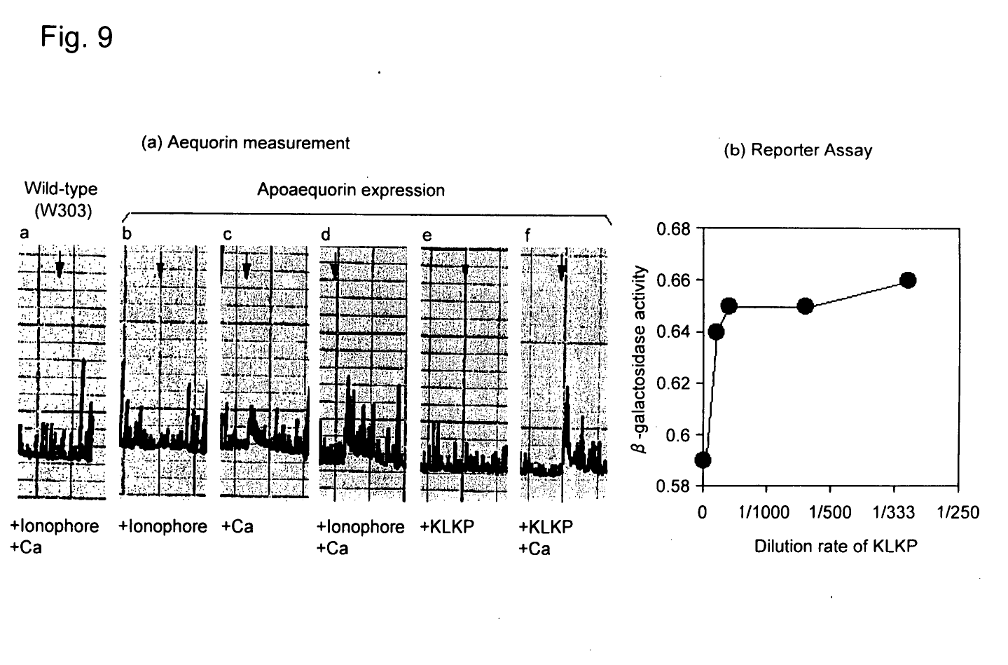

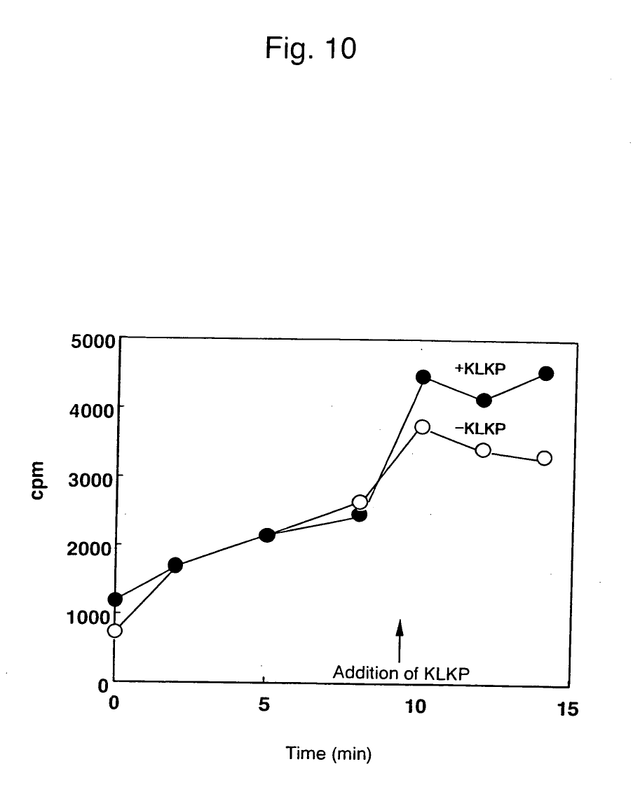

Furthermore, KLKP was caused to act on yeast that had been transformed with a plasmid having a β-galactosidase gene ligated downstream of the promoter of a protein to be induced by Ca²⁺. 5 minutes later, the cells were washed with water. Subsequently, a liquid medium was added to the cells. The resultant was shaken for 4 hours, and then β-galactosidase was expressed corresponding to intracellular Ca²⁺ concentrations. The cells were treated with ether, a substrate (ONPG) was added to the cells, and then the β-galactosidase activity of the cells was examined. The results are shown in FIG. 9(a)a to f (aequorin measurement method), and the β-galactosidase activity, is shown in FIG. 9(b) (reporter assay method). In FIG. 9(a), (a)a shows the results for the wild type, and (a)b to (a)f show the results for the cells expressing the aequorin precursor.

As a result, the cells (FIG. 9 (a)b to f) that had been caused to express the aequorin precursor slightly emitted light due to the addition of Ca²⁺ (FIG. 9(a)c) and the emission levels increased 2.4-fold due to the addition of Ca²⁺ and a calcium ionophore (FIG. 9 (a)d). However, similar to the case of the calcium ionophore, KLKP alone did not induce light emission (FIG. 9(a)e). Nevertheless, the addition of Ca²⁺ and KLKP induced light emission 2.4-fold more intense than that resulting from the addition of Ca²⁺ (FIG. 9(a)f). Thus, it was confirmed that Ca²⁺ in an amount equivalent to that of the calcium ionophore increased within the cells immediately after the addition of KLKP.

Subsequently, reporter assay was conducted. In the case of cells subjected to KLKP treatment, it was considered that intracellular Ca²⁺ concentration increased because of increased β-galactosidase activity (FIG. 9(b)).

By the above two methods, it was confirmed that intracellular Ca²⁺ concentration was increased by KLKP treatment.

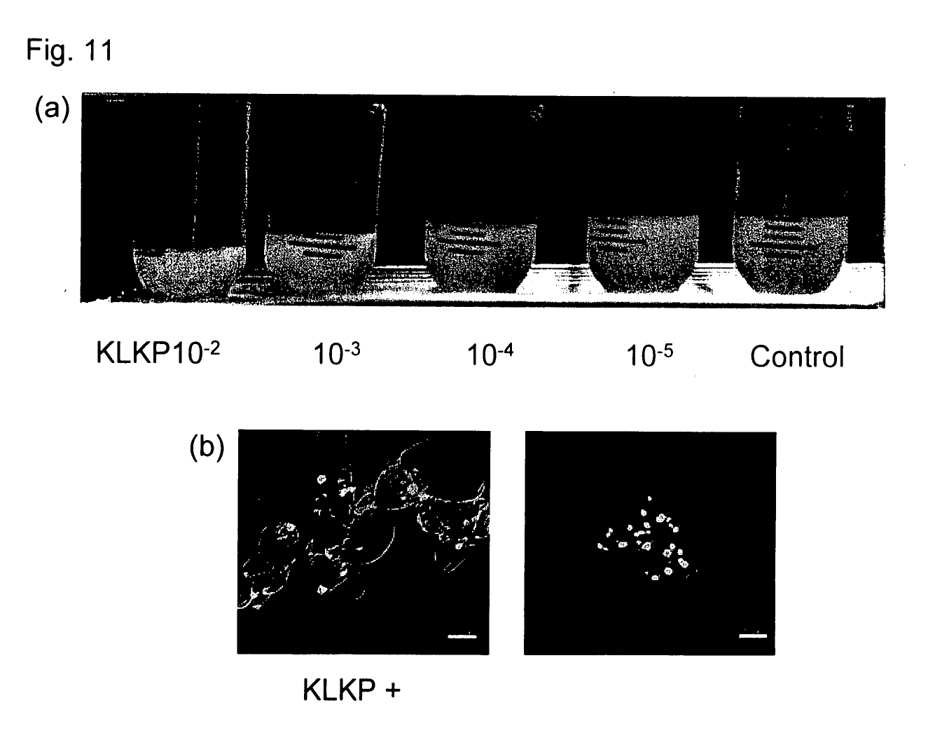

Furthermore, the action of KLKP was analyzed using ⁴⁵Ca. ⁴⁵Ca was added to yeast cells that had been cultured in a medium lacking calcium. 9 minutes later, when the calcium absorption level reached a flat level, KLKP was added. Cells were sampled every 2 minutes after the addition of ⁴⁵Ca, so that the intracellular ⁴⁵Ca level was measured. As a result, the intracellular calcium level after the addition of KLKP increased to a level 1.5-fold to 2-fold greater than the case where no KLKP had been added (FIG. 11).

As shown in FIG. 9 and FIG. 11, the influx of calcium ions from outside of cells due to KLKP was confirmed.

The activation of calcineurin due to KLKP was confirmed by the following methods.

Based on the fact that the calcium-signaling pathway activates Swe1 (FIG. 4), the Δzds1strain was used to enable to observe Swe1 activation due to the Ca²⁺ signaling.

When the calcium-signaling pathway was blocked, killer sensitivity decreased. In particular, in the case of double disruption (where both the calcium-signaling pathway and Cnb1 were disrupted), the double disruption strain came to have enhanced resistance compared with the parent strain, and did not become killer-sensitive without calcineurin.

It is known that the Δzds1 strain is delayed at the G2 phase when calcium is added. As a result of FACS analyses, when KLKP was caused to co-exist with calcium, the strain is further delayed at the G2 phase (FIG. 5). Thus, it was confirmed that the calcium-signaling system in FIG. 4 was activated.

The above results revealed the presence of a mechanism wherein intracellular calcium concentration was increased by KLKP, resulting in an activated calcium-signaling pathway, calcineurin, and a delay at the G2 phase of the cell cycle.

### EXAMPLE 3

### Suppression of Cultured Plant Cell Proliferation by Killer Protein (KLKP) Produced by Kluyveromyces lactis Killer Yeast

1 ml of cultured tobacco cells (BY cells) that had been grown at 28° C. for 7 days was added to 20 ml of a Muraslige and Skoog medium containing 3% sucrose. KLKP was added to the medium at a final concentration between 1/100 and 1/100000, and then cultured for 5 days at 28° C. and 115 rpm. The broth containing cells was transferred into a calibrated test tube, and then allowed to stand. The amount of the precipitated cells was measured. Furthermore, cells to which no KLKP had been added and cells to which KLKP had been added at a concentration of 1/100 were stained with the DAP1 fluorescence reagent, so that the morphology of the cells and the nuclei could be observed.

As a result, in the presence of KLKP at a concentration of 1/100, the proliferation of the culture cells was approximately half of that of the cells to which no KLKP had been added. Even in the presence of KLKP at a concentration of 1/1000, the amount of the culture cells was approximately 80% of that of the cells to which no KLKP had been added, showing that cell proliferation was inhibited by KLKP. Furthermore, the cell size became greater than the general cell size through the addition of KLKP, showing that cell division was inhibited by KLKP (FIG. 11). In FIG. 11, FIG. 11(a) shows increases in cells, and FIG. 11(b) shows changes in cell morphology. In addition, it was revealed that KLKP acts also on eukaryotes other than yeast.

### INDUSTRIAL APPLICABILITY

As shown in Example 2, the killer protein (KLKP) produced by Kluyveromyces lactis killer yeast can be utilized as a calcineurin activator in eukaryotic cells, because the protein causes an increase in intracellular calcium ion concentration and activates calcineurin in eukaryotic cells. Furthermore, KLKP can arrest the cell cycle of eukaryotic cells at the G2 phase in the presence of calcium ions, so that it can be utilized as an agent for inhibiting the cell cycle. In particular, as shown in Example 3, KLKP can suppress proliferation of eukaryotic cells other than yeast, so that it can be used as an agent for inhibiting the proliferation of a variety of cells. In particular, deterioration of silage can be prevented by adding the calcineurin activator of the present invention comprising KLKP as an active ingredient to silage together with a calcium ion supply source.

Furthermore, the calcineurin activator of the present invention comprising KLKP as an active ingredient can be used as a medicine such as an anti-cancer agent.

All publications cited herein are incorporated herein by reference in their entirety. It is easily understood by those skilled in the art that various changes and modifications may be made in the present invention without departing from the technical idea and the scope of the present invention described in the attached claims. It is intended that the present invention encompasses such modifications and changes.

1-11. (canceled)

12. A method of activating calcineurin in a eukaryotic cell, which comprises:
contacting a eukaryotic cell with an isolated Kluyveromyces lactis killer protein (KLKP) in an amount sufficient to activate calcineurin.

13. The method of claim 12, wherein said eukaryotic cell is a mammalian cell.

14. The method of claim 12, wherein said KLKP comprises the α, β, and γ subunits.

15. The method of claim 12, wherein said KLKP comprises an α subunit which is at least 95% homologous to SEQ ID NO: 2, a β subunit which is at least 95% homologous to SEQ ID NO: 3 and a γ subunit which is at least 95% homologous to SEQ ID NO: 4.

16. The method of claim 12, wherein said KLKP is present in an amount sufficient to induce a delay in the G2 phase of the cell cycle.

17. The method of claim 12, wherein said KLKP is present in an amount sufficient to inhibit proliferation of the eukaryotic cell.

18. The method of claim 12, wherein said KLKP is present in an amount sufficient to activate T cell proliferation and said eukaryotic cell is a T cell.

19. The method of claim 12, wherein said KLKP is present in an amount sufficient to induce cancer cell differentiation, cancer cell apoptosis, or reconstruction of myofibrils, and said eukaryotic cell is a cancer cell.

20. The method of claim 12, wherein said eukaryotic cell is a fungus or yeast, and said fungus or yeast is contacted with an amount of KLKP sufficient to inhibit its proliferation.

21. The method of claim 12, wherein said eukaryotic cell is present in silage and an amount of KLKP sufficient to inhibit the deterioration of said silage is added to the silage.

22. A method for treating a disease or disorder, comprising:
administering an effective amount of a KLKP to a subject in need thereof,
wherein said disease or disorder is selected from the group consisting of cancer, a cardiovascular disease, a memory disorder, a disorder requiring immunosuppression, and a disorder requiring immunostimulation.

23. The method of claim 22, wherein said disease or disorder is cancer.

24. The method of claim 22, wherein said disease or disorder is a cardiovascular disease.

25. The method of claim 22, wherein said disease or disorder is a memory disorder.

26. The method of claim 22, wherein said disease or disorder is a disorder requiring immunosuppression.

27. The method of claim 22, wherein said disease or disorder is a disorder requiring immunostimulation.

28. A method for screening for a compound that inhibits calcineurin activity, comprising:
contacting a KLKP-sensitive yeast with a test substance and a Kluyveromyces lactis killer protein (KLKP) which is a calcineurin activator;
determining the ability of the test substance to affect the sensitivity of the KLKP-sensitive yeast to the calcineurin activator, and
selecting a test substance which inhibits the action of the calcineurin activator on the KLKP-sensitive yeast cells.

29. The method of claim 28, further comprising evaluating the ability of said selected test compound to treat a disease or disorder selected from the group consisting of cancer, a cardiovascular disease, a memory disorder, a disorder requiring immunosuppression, and a disorder requiring immunostimulation.


SEQUENCE LISTING (4 sequences)

SEQ ID NO: 1 - DNA, 8874 bp, Kluyveromyces lactis
acacataacataggggagagtactaaaagtgagattattggaagattagtacgtctccatttttttctgtttttttgtttttatatattaggttattttttttcagttttatatcaactctgtataacaagtctatttttttatattttaagtctattttacacttttgacctataagtcattttattatacacattttccaactataatatatgaattacattattaatttaaaaatggattacaaagataaggctttaaatgatctaagaaatgtatatgccgactttgattcacttcctttagattttagacaaatattaataaaagatagagccacacttcttcaaaaagaagatgtagaaaagaaaatattggaaagacaagaagatgcaaagaaatatgcagaatatttaaaacaatcagaaataccagaacgaatatctttgcctaacattaaaagacataaaggtgtttctatatcttttgaagaaacatcagaagatatggttttggaaccaagaccttttatttttgatggattaaatattagatgttttagacgagagacaattttctctctcaaaaataaaatattaaacatggtaaaagaaagttcttcttttaaaaatgtttctagacaatcagtttctttcatgtattttaaaatttttaataaagggaaagttatagcttctacaaaaagtgtaaatatttatgaagataaaatagatgagagattagaagatttgtgtaataattttgacgatgtattaaagaaaattatagatgtaacttatggttatgaaagtttatttgtttcagaaacatattcttatgttatattttatgctaaatctatatatttccctcaacctagatgtgtgaataattggggtaataatattcctaatattcttactttcgatagttttaagcttttcacagctaataaaaataatgtttcttgtattaaacagtgctctcgttttctgtggcaaaaagattttaatacattagaagaaatgatagaatataaaaatggtaatatttgtatagttactcctcaattacatataaatgatgtaagagacataaaatcatttaacgacatacgtttatattcagaaagtcctattaaaacattcagtgttatagataatactataacatatttgttttattttaaagaacatttaggagttatatttaatattactaaatccagacatgatagaagagtcactaaatttagtcctttgtcaaaattttctgatgttaaaaatataacagtatgttttgatatagaatcttattttgatccagaaaaagaatctaatcaagttaatataccctttatatgttgtgcatctataatatataataaagtcataggaaatattgtagattttgaaggaagagattgtgtagctcaaatgatagaatatgttgtagatatatgtggagagcttaatatatcttcagtggaactaattgcacataatggtggaggttatgattttcattatattttaagtagtatgtataatcctgcagctattaaaaatatattaattagaaataactcatttataagttttaattttgctcacgatggagtcaaattttctgtaaaagattcctatagtttcttgttatgtagtttagcaaatgcttcaaaagcatttttaaacgaagaaacctttaagaaaacagattttccccatcatgatttaaaaacagcagatgatttatataaagtatataaagaatggtcatctgtaaacactgaaataaatcatgtagtggaaaaagaaaaacttcttataacatcagaacatatagttaatttcactaaaaatgataaatctaaaactctaatagaatggtctaaagattattgtagaaatgatgttttggttttatctaaggtatggttagaatttaaaaatgctgtagaagatatttttaattgtgaattagtagatcaaactatgacattagcaggactaagttataaattatttcaagcaaatatgccttttgatgttgaattaagacatccaaataaagaagattattttaacatgagagaggctttaataggagggagatgtattagtgtcaatggaatatataaagatgttttatgtttagatgtaaaatcattatatccagcatctatggcattttatgaccagccatatggatctttcaaaagagtatctagtagacctaaagatgaattaggtatttattatgtcagagtaactcctaatagaaataataaatccaacttttttcctataagaagtcacaataaaattacttataataattttgaagaaagtacatatatagcatggtatacaaatgtagatatagatataggtttgtctgaaggtcataatatagaatatatcccctttgattcttatggaaatataggttattcttggtctaaaaaaggtaaaatattcgaaaaatatataaaagacgtgctgtacaaattaaaaataaagtatgaaaaacaaaacaataaagttaaaagaaatgttatcaaaattattatgaacagtttatggggcaaattcgcacaaaaatgggtaaattttgagtattttataaaatcagaagatgatatagattttgagtcagaagaggcatataagatatgggacactgattttatgctgataaagaaaattaaagaatctacttattcatctaaacctatacaaaatggagtatttacattaagttgggcaagataccacatgaaaagtatatgggatgcaggggctaaagaaggagcagaatgtatctattcggacacagatagtatttttgtacataaagaacattttaaaaagaatgctaaatttatgttaaatggtttaaaagttcctattataggatcagaagtaggacaattagaattagaatgtgagtttgataaattgttatgtgcaggtaaaaagcaatacatgggattttatacttattttcaagatggaaaaccatgtataaaagaaaagaaaagatttaagggtattcctagtaattatataatacctgaattatatgctcatttactttcaggtgcagacaaagaagctaaaatacaatttttgaaatttagaagagaatggggatcagttaaaggatatatagaaaataagaccgtgaaagctacttaatatatgaaagtttttataataattataaaatgaatatattttacatatttttgtttttgctgtcattcgttcaaggtttggagcatactcatcgaagaggctccttagtcaaaagagcagtatgttatgacactgatcaagttccacttaatattttctttggttataatagagcagataagactgattctaataagaatatggctctaaacatctttaatgtttttagaggttttctagctggagaaggtggagagtctttttacaattctaatggtaatgtttatggatttatgtgggtaggtagtatggttcataatagaggttttaaagataatattttacctataatggaaaatgaagttaagaattatggtattcctaaaaccttgtatttagaatatgacggaggtggagatcctatgaaatcttttggtattattttagatacaacaagtagagatactgtagttaaagctgcaaaattatggagtcaaggtaaaaaattaaatagttatgaaggatctaaaaattatcaagctactgcatgctatttatcttatgcatatagaaagcccattgttaatgataattttgtaggaacttgcgactatttcactttagaaagtggtaaaacaccagcagaccaatctggtattaatggagagtctctacaaggttataatcctaatttagatttctctaaattatcagcaggacaacctatttgtaaaaccataggtaatcctcctaattttaaaccttctaagaattcagacggttcttgtaaaacatacaaggtatcatctggagagtcttgttcttctatagcagttaaatattatccattaagtttaaatgatatagaaaattataataaaggtaattatggatggaaaggatgttctagtcttcaaaaagattataacttatgtgtgagtgatggtagtgctcctagaccagtttcaaatcctatagcagaatgtggtccattagctccaggagagaaatataatgctaaatgtcctttaaatgcttgttgtagtgaatttggtttctgtggtttaactaaagattattgtgacaaaaagagtagtactactggtgctcctggtacagatggctgtttttctaattgtggttatggttctacttctaatgtaaaatcatctacttttaaaaagattgcttattggttagatgctaaagataaattagctatggatccgaagaatattcctaatggtccttatgatattttacattatgcttttgttaatataaattcagactttagtattgatgattctgcattttcaaaatctgcctttttaaaagttacttcttccaaaaagatacctagttttggtggttgggattttagtacatctcctagtacttacactatatttagaaatgctgttaaaacagatcaaaatagaaatacgtttgctaacaatttaatcaattttatgaataaatataatcttgatggtatagatttagattgggaatatccaggtgctcctgatattccagatattcctgctgatgattcaagtagtggatctaattatctaactttccttaagttattaaagggtaaaatgccttctggtaaaaccttatctatagccattccttcttcctattggtatttaaaaaatttccctatttctgatattcaaaacactgtagattatatggtttacatgacgtatgatatacatggtatatgggaatacggtaaagccaatagttatataaactgccatactcctcgtaaagaaattgaagatgctataaaaatgttagataaagctggagttaaatttaataaagtatttggaggtgtggcaaattacggtagatcctacaaaatggttaatacaaattgttataattatggatgcggttttcaaagagagggaggaaattctagagatatgactaatacaccaggtgttctttctgattcagaaattattgatattgatagttcagataaaaagaatgatagatgggtagatactaacacagattgtatttttatgaaatatgacggaaattctgttgtttcatggcctaaaagtagatacgatttagaagatatgtttaaaaattatggatttgctggtacttctttatgggccgctaattatttcaaacatgatgaatggaagaacgatgaagatgataataatgatgatacagaagatcctttcgatgaagagaatgtatatttcgatgtttatgattgcaaaaacaaagctggttatgatctggacaatccagtttatgggtgtagattagaaacagctataaatattattatatggaatggtacagaatctgttaatacagttttaaatatattaaatgattacgataattatattaaatattatgaagctctaactagagcacattatgattcagtcatggaaaaatacgaaaaatggctgtttgaagaagatggatattacacatattatactgatgtagacggagatgatataattataactcctccagataagaagaaaagagattacatacaagagaaatattcttttgaaaaagaatttatgatgtctcaaaatatgacagaattaacagaaattaaagttaataaaactattaattttatgttaaatggaacatctctagctgtaaaagaatataacaacgaaaaagttttatataaaagaggagatatacctcctcctggttctaataatagattaattagaaacagtattattttagataaagataaagaagcagctattgcgtctttcaaacaatattctggaatagaattatctaaagattcttttgtacaaagagataaagataaaaagtttgatctaaatggtaaacattatacatttatgcatagtactattctgaatgctattgttttattccctaatgttttaacaaatattgattctgactatattcatcatatttcagatttaattgaacaagctcataacagtttaggtaatgaaagtcctgataatatttatgaggtcttagaaagtgtggttgtttttatgtctgtatcagaaatagctgattatacatatacagaaggtaaaaagataaaagaaaaatacgataagatgaagaaaactatgattgttggtattatattgggtatcataggtggtttgtctctatttttaggacctataggtatagctacatctgttcttgcagattttgctctattaggagcagatgccgctataaacggagagttaaatccatcagacctagcattcgctttagcaggtttattcttaccagtatttgcttctttaggaaaaacatttaaatttgctgaagctttacaaaaaattaatattaataaatctaaaaactttgataatttaaatgaatttgagaaaataagatttttcagatctaaattagggaaagttaagatgtgtggctcttaaaagtaatggatgaccattattcttgtgtaaattgtcaaaatctacatcttcatatttatgatatttaaatatatatttttcgttttcaaaatctaaatgttgacacatacctccttctttttttgctttattcatcataatattataaaattcaatactaccagaagcataagctattcttattaaatctatatctggactataattttctaaatcttcagttatattcataatagcataatttactaatattgcatatctttggcgtggaaaatcgataagtagtttttgaaccatatatttatttaaagttttataagtgtaaaaataaaaaggcctataaagagacacaaagtttgaatcataaatatcattcactaataaatttaatactgcttttttacacaaatcatctggatattctttatgatgtttaagtacataagctgaatttaaaaaattaaattcaactgtatttatatttatatctaaataaggtttataagagaccatattatagtacacacttttatctacagaaacacaatccataggaccaaattctgtattttgactataatctatatatgtatataacatatcatctataatttgttctatattactttgtttagaagtataattatatttaaaaaatatttctaaagttgtgtctttattcctgagtatagtttcaggaagtaaatatttgtctttttctactttttctaaaatatttttattttcatgtattttataattatatatagtatcttcttcgcaaaaagatcttctattaaaaattatagataatctaaaacaaacttctgtacatattttatcacatttatcacaaacatcatcccaacctaataaaacacatattgttttaataactaaactatatttaggatctttttctaataaatatatacaagtttctttagtaggaacattaggataccaaattcctgaaggcaaatatttaaaattaaaatcacaaaccttgttactcataatatatctagcagatatactggaaagtattccagatgttaattttaaacctgaattttccattttaactgcaaaattataactatttcttattcctatacataaatgaaagtttaaatcatcttcatcaaaaagctcttcattatcataatatttaataaaattttcataatctgaataataagcataagtacatgctttaaaataatctgaaagattattatctaattctaaacacatttttaattaaaatgaagatatatcatatatttagtgtttgttatctaataacattatgtgctgctgcagctactactgcgagagaggagtttttcttatgttatgatttaattagatatttaaaacaatatgaaaaaacaggagagagtaaattagtagaacaaacattttttaatagtattaaaaacttagacataaactctagagagtatatggaacttgtatataacaaaatagcaggtatttccaatgaaagaaataaatttgaaaatatatataaagatggagattctataagtcaagttgtagaaagagctgtaagcgaaaagaaacttacatttggattaaacggtaaaggattatatgttccagaaaacggagaaccccgactaaaaggttatgcttctattatagaaagaataactctggatttaatggaaatatattctattaaaggacttaatgatatacctagagatataaaatttaatatggaaaaaataagacaagaaagatacaaccaaatgaaagaagctctaaatagtgttgaaggttataaaggaaaaattgtagcctcagactcagattggtgtttcaaagatcctcaaggcaatagaataacagattttgatagtattaataaagaattaggtcttggtagaagagatgtaaaattagataaaggtcatgatgatttaattaaattatgtactgaaaaaatagatagtatgaataatctacagaatggaaaatgtgtataataaaatgacttataggtcaaaagtgtaaaatagacttaaaatataaaaaaatagacttgttatacagagttgatataaaactgaaaaaaaataacctaatatataaaaacaaaaaaacagaaaaaaatggagacgtactaatcttccaataatctcacttttagtactctcccctatgttatgtgt

SEQ ID NO: 2 - protein, 863 aa, Kluyveromyces lactis
AVCYDTDQVPLNIFFGYNRADKTDSNKNMALNIFNVFRGFLAGEGGESFYNSNGNVYGFMWVGSMVHNRGFKDNILPIMENEVKNYGIPKTLYLEYDGGGDPMKSFGIILDTTSRDTVVKAAKLWSQGKKLNSYEGSKNYQATACYLSYAYRKPIVNDNFVGTCDYFTLESGKTPADQSGINGESLQGYNPNLDFSKLSAGQPICKTIGNPPNFKPSKNSDGSCKTYKVSSGESCSSIAVKYYPLSLNDIENYNKGNYGWKGCSSLQKDYNLCVSDGSAPRPVSNPIAECGPLAPGEKYNAKCPLNACCSEFGFCGLTKDYCDKKSSTTGAPGTDGCFSNCGYGSTSNVKSSTFKKIAYWLDAKDKLAMDPKNIPNGPYDILHYAFVNINSDFSIDDSAFSKSAFLKVTSSKKIPSFGGWDFSTSPSTYTIFRNAVKTDQNRNTFANNLINFMNKYNLDGIDLDWEYPGAPDIPDIPADDSSSGSNYLTFLKLLKGKMPSGKTLSIAIPSSYWYLKNFPISDIQNTVDYMVYMTYDIHGIWEYGKANSYINCHTPRKEIEDAIKMLDKAGVKFNKVFGGVANYGRSYKMVNTNCYNYGCGFQREGGNSRDMTNTPGVLSDSEIIDIDSSDKKNDRWVDTNTDCIFMKYDGNSVVSWPKSRYDLEDMFKNYGFAGTSLWAANYFKHDEWKNDEDDNNDDTEDPFDEENVYFDVYDCKNKAGYDLDNPVYGCRLETAINIIIWNGTESVNTVLNILNDYDNYIKYYEALTRAHYDSVMEKYEKWLFEEDGYYTYYTDVDGDDIIITPPDKKKRDYIQEKYSFEKEFMMSQNMTELTEIKVNKTINFMLNGTSLAVKEYNNEKVLY

SEQ ID NO: 3 - protein, 252 aa, Kluyveromyces lactis
GDIPPPGSNNRLIRNSIILDKDKEAAIASFKQYSGIELSKDSFVQRDKDKKFDLNGKHYTFMHSTILNAIVLFPNVLTNIDSDYIHHISDLIEQAHNSLGNESPDNIYEVLESVVVFMSVSEIADYTYTEGKKIKEKYDKMKKTMIVGIILGIIGGLSLFLGPIGIATSVLADFALLGADAAINGELNPSDLAFALAGLFLPVFASLGKTFKFAEALQKININKSKNFDNLNEFEKIRFFRSKLGKVKMCGS

SEQ ID NO: 4 - protein, 249 aa, Kluyveromyces lactis
MKIYHIFSVCYLITLCAAAATTAREEFFLCYDLIRYLKQYEKTGESKLVEQTFFNSIKNLDINSREYMELVYNKIAGISNERNKFENIYKDGDSISQVVERAVSEKKLTFGLNGKGLYVPENGEPRLKGYASIIERITLDLMEIYSIKGLNDIPRDIKFNMEKIRQERYNQMKEALNSVEGYKGKIVASDSDWCFKDPQGNRITDFDSINKELGLGRRDVKLDKGHDDLIKLCTEKIDSMNNLQNGKCV